(19) United States
(12) Patent Application Publication
(10) Pub. No.: US 2023/0189846 A1
(43) Pub. Date: Jun. 22, 2023

(54) Novel Silage Additive Compositions

(71) Applicant: Locus IP Company, LLC, Solon, OH (US)

(72) Inventor: Sean FARMER, Ft. Lauderdale, FL (US)

(21) Appl. No.: 17/765,614

(22) PCT Filed: Jul. 23, 2021
(86) PCT No.: PCT/US2021/042921

§ 371 Date: Mar. 31, 2022

Related U.S. Application Data
(60) Provisional application No. 63/056,663, filed on Jul. 26, 2020.

(51) Int. Cl. A23K 30/18; A23K 10/18; A23K 20/28; A23K 10/30; C12N 1/20; C12P 7/56; A23K 50/10

(52) U.S. Cl. CPC A23K 30/18; A23K 10/18; A23K 20/28; A23K 10/30; C12N 1/20; C12P 7/56; A23K 50/10; C12N 2500/02; C12R 2001/07

(57) ABSTRACT

Compositions and methods are provided for producing silage additive composition for producing silage and for controlling pests that infect silage and stored grains. The silage additive composition comprises a microbial inoculant and diatomaceous earth, wherein the microbial inoculant comprises one or more strains of bacteria, wherein the at least one of one or more strains bacteria is capable of producing lactic acid.

### CROSS REFERENCE TO RELATED APPLICATION

This application claims priority to U.S. Provisional Patent Application No. 63/056,663, filed Jul. 26, 2020, which is incorporated herein by reference in its entirety.

### BACKGROUND OF THE INVENTION

Silage is a type of fodder made from green foliage crops that have been preserved for feeding cattle, sheep and other livestock animals year-round. The silage must be protected from arthropod pests, such as, for example, aphids, earworms, rootworms, cutworms, grasshoppers, mites, slugs and wireworms, as well as spoilage from microbial pests.

The “ensiling” process is a method of moist forage preservation and is used all over the world. Cut vegetation is placed in a silo or pit, compressed to remove as much oxygen as possible, and covered using, for example, a lid, tarp or plastic sheet. Alternatively, large round bales of vegetation can be wrapped tightly in a plastic film, which compresses the vegetation inward.

After the compression and sealing of the vegetation, ensiling involves natural fermentation, where lactic acid bacteria (LAB) convert water soluble carbohydrates in the vegetation to organic acids under anaerobic conditions. This causes a decrease in pH, which then inhibits detrimental microbes so that the moist forage is preserved.

More specifically, the ensiling process can be characterized by four different phases. First, upon sealing, oxygen is still present between plant matter particles and the pH is only slightly acidic, e.g., about 6.0 to 6.5. These conditions allow for continued plant respiration, protease activity and activity of aerobic and facultative aerobic microorganisms. The second phase comprises fermentation, which begins after the silage becomes anaerobic and lasts several days to several weeks. LAB grow and become the primary microbial population, thereby producing lactic acid and other organic acids, decreasing the pH to, e.g., about 3.8 to 5.0.

The third phase is stable, with few changes occurring in the characteristics of the forage so long as air is prevented from entering the silage environment. The final phase is feedout, when the silage is ultimately unloaded and exposed to air. This results in reactivation of aerobic microorganisms, primarily yeast, molds, and certain bacteria, which can cause spoilage.

Aerobic instability is a primary problem in silage production. Even before storage units are open for feedout, silage can be exposed to oxygen because of, e.g., poor packing or sealing. Under these types of aerobic conditions, rapid growth of yeast and mold cause silage to heat and spoil, decreasing its nutritional value. Aerobic instability can be a problem even in inoculated silage that has undergone what would traditionally be considered an ideal fermentation phase, meaning a rapid drop in pH and a low terminal pH. In such conditions, yeasts that contribute to spoilage are, for example, tolerant of acidic conditions and/or capable of metabolizing lactic acid produced by LAB during fermentation.

While taking care in packing silage effectively and in removing silage for animal feed can help minimize the aeration of the silage environment, additional methods are often needed to prevent spoilage. Some methods utilize chemical and/or biological additives. These include, for example, fermentation stimulants such as molasses, sucrose, glucose, citrus pulp, pineapple pulp and sugar beet pulp; enzymes, such as cellulases, hemicellulases, pectinase, and amylases; and inoculants such as lactic acid bacteria (LAB). Additives also include fermentation inhibitors, for example, acids and organic acids, such as hydrochloric acid, formic acid, acetic acid, lactic acid, acrylic acid, calcium formate, propionic acid, and propionates; and chemicals such as formaldehyde, sodium nitrite and sodium metabisulphite.

Many of the chemical-based additives, such as formic acid, formaldehyde, propionic acid and ammonia can be unsafe to handle and can be corrosive to equipment. Thus many growers prefer to utilize biological additives. Bacterial inoculants, in particular, have advantages over chemical additives because they are safe, easy to use, non-corrosive to farm machinery, they do not pollute the environment and are regarded as natural products.

Silage inoculants containing “homofermentative” LAB are commonly used as biological additives in many parts of the world. Homofermentative LAB include, for example, certain members of the genera Lactococcus, Lactobacillus, Streptococcus, Propionobacter, Enterococcus, Pediococcus and Aerococcus. Their function is to produce lactic acid and a rapid decrease in pH through fermentation of a crop's water soluble carbohydrates. Inoculants can also reduce aerobic spoilage and improve animal performance as part of feed.

The use of “heterofermentative” LAB, which include, for example, certain Leuconostoc, Propionibacter, and Lactobacillus spp., has recently gained favor as well. Heterofermenters have the ability to ferment glucose into multiple different by-products. These include, for example, lactic acid, ethanol and carbon dioxide. They can also convert lactic acid to acetic acid and/or propionic in the presence of oxygen, and the acetate produced may inhibit other deleterious organisms, such as clostridia and fungi. One downside of using heterofermentative LAB, however, is the loss of significant portions of dry matter, which is consumed and converted to carbon dioxide gas.

While bacterial silage inoculants are an important aspect of grain and silage storage, their use can be greatly limited by difficulties in production, transportation, administration, pricing and efficacy. For example, many microbes are difficult to grow and subsequently deploy, e.g., into a silage storage tank, in sufficient quantities to be useful. This problem is exacerbated by losses in viability and/or activity due to processing, formulating, storage and stabilizing prior to distribution. Furthermore, once applied, biological products may not thrive for any number of reasons including, for example, insufficient initial cell densities, the inability to compete effectively with the existing microflora at a particular location, and being introduced to silage and/or other environmental conditions in which the microbe cannot flourish or even survive.

The ensiling process is a complex one and involves interactions of numerous different chemical and microbiological processes. Thus, improved compositions and methods for maintaining the aerobic stability and preventing spoilage of silage are needed.

### BRIEF SUMMARY OF THE INVENTION

The subject invention provides microbe-based products for use in silage production and grain preservation. Specifically, the subject invention provides silage additives for producing silage and for controlling pests that infest silage and stored grains. Advantageously, the microbe-based products and methods of the subject invention are environmentally-friendly, non-toxic and cost-effective.

In preferred embodiments, the subject invention provides silage additive compositions comprising one or more microbial inoculants. Advantageously, in certain embodiments, the production and preservation of silage through fermentation, using the subject microbial inoculants can prevent spoilage of silage, control pests, and/or enhance the nutritional benefits of silage as a livestock feed product.

In one embodiment, the microbial inoculants as described herein can include one or more bacterial cultures comprising a bacterial strain and a growth by-product thereof, such as, for example, an organic acid, a biosurfactant and/or an enzyme. Preferably, the microbial inoculant supplies at least 20 billion, at least 30 billion, or at least 40 billion CFU of a microorganism per gram of forage to which the composition will be applied.

In some embodiments, the microbial inoculant can comprise nutrients and/or substrate leftover from cultivation of the microbial inoculant. The microbes can be live or inactive, although in preferred embodiments, they are live. The inoculants may be in the form of, for example, a fresh live culture, rehydrated lyophilized bacterial cells or spores, or a thawed frozen bacterial preparation.

In preferred embodiments, at least one of the bacterial species is a type of lactic acid bacteria (LAB), for example, a Lactococcus, Lactobacillus, Streptococcus, Propionobacter, Enterococcus, Pediococcus, Aerococcus, or other LAB.

In certain specific embodiments, the LAB is a bacterium not traditionally categorized as a LAB, but which produces lactic acid as a primary or secondary metabolite. For example, in certain embodiments, the LAB is a strain of Bacillus spp., such as B. coagulans.

In some embodiments, B. coagulans is capable of producing lactic acid and other organic acids. Additionally, in some embodiments, B, coagulans is more thermotolerant than other species of LAB (e.g., capable of growth at 50 to 52° C.), which can increase the effectiveness of the ensiling process in hotter climates and/or in the event of an air leak in the sealed silage environment.

In some embodiments, a bacterial species or strain capable of producing other beneficial growth by-products, such as other organic acids, biosurfactants and/or enzymes helpful for preservation of silage and/or pest control. In certain specific embodiments, a strain of B. amyloliquefaciens is used, e.g., B. amyloliquefaciens strain NRRL B-67928 (“B. amy”). In certain specific embodiments, a strain of B. subtilis is used, e.g., B. subtilis strain NRRL B-68031 (“B4”). In some embodiments B. amy and B4 can be used together.

In some embodiments, B. amy is capable of producing antifungal, antiviral and antibacterial lipopeptide biosurfactants, organic acids such as propionic acid and acetic acid, and enzymes, such as amylases, lipases and proteases.

In some embodiments, B4 is capable of producing the antimicrobial lipopeptide surfactin in enhanced amounts over reference strains of B. subtilis. Thus, in some embodiments, B4 is a surfactant “over-producer.”

In some embodiments, a combination of B. coagulans and B. amy and/or B4 allows for enhanced silage fermentation and direct control of microbial pests in a silage or grain storage environment.

In one embodiment, the composition comprises B. coagulans and B. amy, wherein B. coagulans comprises 1 to 99% by weight or volume of the microbial inoculant portion of the composition and B. amy comprises 99% to 1% by weight or volume.

In one embodiment, the composition comprises B. coagulans and B4, wherein B. coagulans comprises 1 to 99% by weight or volume of the microbial inoculant portion of the composition and B4 comprises 99% to 1% by weight or volume.

In one embodiment, the composition comprises B. coagulans with B4 and B. amy, wherein B. coagulans comprises 1 to 99% by weight or volume of the microbial inoculant portion of the composition, and the combination of B. amy and B4 comprises 99% to 1% by weight or volume.

The species and ratio of microorganisms and other ingredients in the composition can be determined according to, for example, the type of forage or grain being treated, the humidity/moisture levels, and other environmental factors. Thus, the composition can be customizable for any given forage crop.

In certain embodiments, the silage additive composition comprises additional components, for example, carriers, dessicants, salts, dyes, flavors, buffers, nutrients, starches, carbohydrates, water, and/or additional microbial cultures and/or fermentation by-products.

In certain embodiments, the silage additive composition comprises diatomaceous earth, which can serve as, e.g., a pest control agent for silage and grain pests that have exoskeletons. Advantageously, diatomaceous earth can also serve as a hydroscopic substance, or desiccant, for reducing condensate formation in a silage environment or grain storage unit, thereby producing an environment less favorable for mold and fungal growth.

In some embodiments, the silage additive composition comprises the fermentation products of one or more additional microorganisms, including, for example, beneficial yeasts, fungi and/or other bacteria. These microbes can include, for example, Wickerhamomyces anomalus, Starmerella bombicola, Saccharomyces chlororaphis, Saccharomyces cerevisiae, Pleurotus ostreatus, Debaryomyces hansenii, and/or Meyerozyma guilliermondii. The fermentation product can comprise, for example, purified or crude form growth by-products of the beneficial microorganism, fermentation medium, cells, and/or nutrients residual from fermentation. In an exemplary embodiment, the composition comprises a crude or purified form biosurfactant, such as a sophorolipid, produced by a beneficial microorganism.

In preferred embodiments, methods for ensiling forage are provided, wherein the method comprises inoculating forage with a silage additive composition according to the subject invention, and wherein the microbial inoculant(s) of the composition grow anaerobically, thereby fermenting the forage and producing silage. The forage can comprise cuttings of, for example, grass, maize, sorghum, cereals, oats, and/or alfalfa.

Inoculation with the subject composition can comprise applying the composition to the forage and, optionally, mixing the composition with the forage. Preferably, the forage is contained in a suitable “silage environment,” meaning, for example, an airtight silage bag, tarp, silo or tank, which can be sealed after inoculation with optional mixing so that anaerobic fermentation can occur.

In certain embodiments, the silage additive composition is applied directly to the forage in dried form, after which a source of liquid and/or nutrient medium is applied to the mixture to create an environment conducive to fermentation. In certain other embodiments, when the forage has a sufficient moisture content, e.g., 45% to 90% moisture content by weight, the silage additive composition can be applied in dried form without the addition of a liquid. In yet other embodiments, the silage additive is pre-mixed with water or a nutrient medium before application to the forage.

In some embodiments, fermentation of the forage proceeds for a period of time ranging from, for example, about 24 hours to 12 weeks, or about 10 days to 3 weeks. The methods can comprise sampling the silage to, for example, monitor the pH throughout the fermentation period and/or to check for potential contamination of the silage. Care should be taken when sampling, however, to avoid introducing air into the silage environment.

In some embodiments, the subject invention provides methods of controlling stored grain pests, such as, for example, grain borers, weevils, moths, and beetles, wherein a silage additive composition of the subject invention is applied to the stored grain. The grain can include, for example, flour, maida, suji, beans, cotton seeds, corn, rice, barley, amaranth, buckwheat, bulgur wheat, farro, flax, kamut, millet, quinoa, rye, spelt, teff, and triticale.

In certain embodiments, application of the silage additive composition provides one or more of the following benefits: enhancing the rate of silage fermentation; controlling molds, fungi and bacteria that can cause spoilage of silage and/or stored grains; controlling arthropod pests that feed on and/or infect silage and/or stored grains; and enhancing the nutritional benefits of the silage and/or stored grains as a feed source for livestock.

In some embodiments, the subject invention provides methods of feeding a livestock animal, wherein silage that has been produced according to embodiments of the subject invention is made available to the livestock animal so that the animal may ingest the silage. Advantageously, the feed source can provide health benefits to the livestock animal, including, for example, improved growth rate (e.g., average daily gain), improved feed-to-muscle conversion, improved milk quality and quantity, increased lifespan, improved immune health, improved gut flora, reduced digestive pathogens, and reduced enteric methane production.

### DETAILED DESCRIPTION OF THE INVENTION

The subject invention provides microbe-based products for use in silage production and grain preservation. Specifically, the subject invention provides silage additives for producing silage and/or for controlling pests that infest silage and stored grains. Advantageously, the microbe-based products and methods of the subject invention are environmentally-friendly, non-toxic and cost-effective.

### Selected Definitions

As used herein, a “biofilm” is a complex aggregate of microorganisms, wherein the cells adhere to each other and/or to surfaces. In some embodiments, the cells secrete a polysaccharide barrier that surrounds the entire aggregate. The cells in biofilms are physiologically distinct from planktonic cells of the same organism, which are single cells that can float or swim in liquid medium.

As used herein, the term “control” used in reference to a pest means killing, disabling, immobilizing, or reducing population numbers of a pest, or otherwise rendering the pest substantially incapable of reproducing and/or causing harm.

As used herein, an “isolated” or “purified” compound is substantially free of other compounds, such as cellular material, with which it is associated in nature. “Isolated” in the context of a microbial strain means that the strain is removed from the environment in which it exists in nature or in which it was produced. The isolated strain may exist as, for example, a biologically pure culture, or as spores or other forms of propagule.

As used herein, a “biologically pure culture” is a culture that has been isolated from materials with which it is associated in nature or in which it was produced. In a preferred embodiment, the culture has been isolated from all other living cells. In further preferred embodiments, the biologically pure culture has advantageous characteristics compared to a culture of the same microbe as it exists in association with the other materials. The advantageous characteristics can be, for example, enhanced production of one or more growth by-products.

In certain embodiments, purified compounds are at least 60% by weight the compound of interest. Preferably, the preparation is at least 75%, more preferably at least 90%, and most preferably at least 99%, by weight the compound of interest. For example, a purified compound is preferably one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, “forage” refers to plant material cut for agricultural purposes, such as feeding livestock animals. Forage plants include, for example, grasses such as bentgrasses, bluestems, false oat-grass, hurricane grass, Surinam grass, koronivia graa, bromegrasses, buffelgrass, Rhodes grass, bermudagrass, orchard grass, antelope grass, bungoma grass, fescues, black spear grass, marsh grass, jaragua, southern cutgrass, ryegrasses, Guinea grass, molasses grass, carabao grass, dallisgrass, reed canarygrass, bluegrasses, bristlegrass, wheatgrass; legumes, such as pinto peanut, peas, trefoil, sweetclovers, perennial soybean, clovers, vetches, vigna, mulga, silk trees, Belmont siris, lebbeck, earpodtree, leadtree; and others, such as alfalfa, maize, sorghums, oats, corn and/or soybean stover.

A “metabolite” refers to any substance produced by metabolism (e.g., a growth by-product) or a substance necessary for taking part in a particular metabolic process. Examples of metabolites include, but are not limited to, biosurfactants, biopolymers, enzymes, acids, solvents, alcohols, proteins, vitamins, minerals, microelements, and amino acids.

As used herein, a “pest” is any organism, other than a human, that is destructive, deleterious and/or detrimental to humans or human concerns (e.g., silage production and/or grain storage). In some, but not all instances, a pest may be a pathogenic organism. Pests may cause or be a vector for infections, infestations and/or disease, or they may simply feed on or cause other physical harm to cut plant matter and/or living tissue. Pests may be single- or multi-cellular organisms, including but not limited to, viruses, fungi, bacteria, molds, protozoa, parasites, arthropods and/or nematodes.

As used herein “preventing” or “prevention” of a situation or occurrence means delaying, inhibiting, suppressing, forestalling, and/or minimizing the onset, extensiveness or progression of the situation or occurrence. Prevention can include, but does not require, indefinite, absolute or complete prevention, meaning the situation or occurrence may still develop at a later time. Prevention can include reducing the extensiveness or severity of the onset of such a situation or occurrence, and/or inhibiting the progression of the situation or occurrence to one that is more extensive or severe.

Ranges provided herein are understood to be shorthand for all of the values within the range. For example, a range of 1 to 20 is understood to include any number, combination of numbers, or sub-range from the group consisting of 1, 2, 3, 4, 5, 6, 7, 8, 9, 10, 11, 12, 13, 14, 15, 16, 17, 18, 19, 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduce” refers to a negative alteration, and the term “increase” refers to a positive alteration, each of at least 1%, 5%, 10%, 15%, 20%, 25%, 30%, 35%, 40%, 45%, 50%, 55%, 60%, 65%, 70%, 75%, 80%, 85%, 90%, 95% or 100%.

As used herein, “surfactant” refers to a surface-active compound that lowers the surface tension (or interfacial tension) between phases. Surfactants act as, e.g., detergents, wetting agents, emulsifiers, foaming agents, and dispersants. A “biosurfactant” is a surface active and/or amphiphilic molecule produced by a living cell and/or using naturally-derived substrates.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Silage Additive Compositions

In preferred embodiments, the subject invention provides silage additive compositions comprising one or more microbial inoculants. Advantageously, in certain embodiments, the fermentation of forage using the subject compositions can prevent spoilage of silage, control pests, and/or enhance the nutritional benefits of silage as a livestock feed product.

As used herein, “microbial inoculants” are compositions that comprise components that were produced as the result of the growth of microorganisms or other cell cultures. Thus, the microbial inoculants may comprise the microbes themselves and/or by-products of microbial growth. The microbes may be in a vegetative state, in spore or conidia form, in hyphae form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. In preferred embodiments, the microbes are present, with growth medium in which they were grown, in the microbial inoculant at, for example, a concentration of at least 1×10³, 1×10⁴, 1×10⁵, 1×10⁶, 1×10⁷, 1×10⁸, 1×10⁹, 1×10¹⁰, 1×10¹, 1×10¹² or 1×10¹³ or more CFU per gram or per ml of the composition.

In one embodiment, the microbial inoculants as described herein can include one or more bacterial cultures comprising a bacterial strain and a growth by-product thereof, such as, for example, an organic acid, a biosurfactant and/or an enzyme.

In some embodiments, the microbial inoculants can comprise nutrients and substrate leftover from fermentation. The microbes can be live or inactive, although in preferred embodiments, they are live. The bacterial cultures may be in the form of, for example, a fresh live culture, rehydrated lyophilized bacterial cells or spores, or a thawed frozen bacterial preparation.

The microorganisms useful according to the subject invention can be, for example, non-plant-pathogenic strains of bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In certain embodiments, the microorganisms are LAB. LAB are Gram-positive, non-spore-forming cocci, coccobacilli or rods. They are generally non-respiratory and lack catalase. They ferment glucose primarily to lactic acid, and/or to lactic acid, CO₂ and ethanol. All LAB grow anaerobically, but some can grow in the presence of CO₂ as “aerotolerant anaerobes.”

Although many genera of bacteria produce lactic acid as a primary or secondary end-product of fermentation, the term Lactic Acid Bacteria is conventionally reserved for genera in the order Lactobacillales, which includes Lactobacillus, Leuconostoc, Pediococcus, Lactococcus, Streptococcus, Cornobacterium, Enterococcus, Propionobacter, Aerococcus Oenococcus, Tetragenococcus, Vagococcus, and Weisella.

In certain specific embodiments, the LAB of the subject compositions is a bacterium not traditionally categorized as a LAB, but which produces lactic acid as a primary or secondary metabolite. For example, in certain embodiments, the LAB is a strain of Bacillus spp., such as B. coagulans.

In some embodiments, B. coagulans is capable of producing lactic acid and other organic acids. Additionally, in some embodiments, B. coagulans is more thermotolerant than other species of LAB (e.g., capable of growth at 50 to 52° C.), which can increase the effectiveness of the ensiling process in hotter climates and/or in the event of an air leak in the sealed silage environment.

In some embodiments, a bacterial species or strain capable of other beneficial growth by-products, such as other organic acids, biosurfactant and/or enzymes helpful for production of silage and/or pest control.

In certain other specific embodiments, a strain of B. amyloliquefaciens is used, e.g., B. amyloliquefaciens strain NRRL B-67928 (“B. amy”).

In certain specific embodiments, a strain of B. subtilis is used, e.g., B. subtilis strain NRRL B-68031 (“B4”).

A culture of the B. amyloliquefaciens “B. amy” microbe and of the B. subtilis “B4” microbe has been deposited with the Agricultural Research Service Northern Regional Research Laboratory (NRRL), 1400 Independence Ave., S.W., Washington, D.C., 20250, USA. The B. amy deposit has been assigned accession number NRRL B-67928 by the depository and was deposited on Feb. 26, 2020. The B4 deposit has been assigned accession number NRRL B-68031 by the depository and was deposited on May 6, 2021.

Each of the subject cultures has been deposited under conditions that assure that access to the culture will be available during the pendency of this patent application to one determined by the Commissioner of Patents and Trademarks to be entitled thereto under 37 CFR 1.14 and 35 U.S.C 122. Each deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposits will be stored and made available to the public in accord with the provisions of the Budapest Treaty for the Deposit of Microorganisms, i.e., they will be stored with all the care necessary to keep them viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposits should the depository be unable to furnish a sample when requested, due to the condition of the deposit. All restrictions on the availability to the public of the subject culture deposits will be irrevocably removed upon the granting of a patent disclosing it.

In some embodiments, B. amy is capable of surviving under conditions of high salt, high heat and high pressures, such as those often utilized in producing compressed salt compositions. For example, B. amy spores can, in some embodiments, exhibit resistance to temperatures of at least 55° C. to 100° C., or at least 80° C. to 125° C.; pressures of at least 200 MPa to 300 MPa, or at least 250 MPa to 350 MPa; and salt concentrations of, for example, 1-15% or higher, e.g., at least 5%, 10%, 12%, 15% or more. In some instances, B. amy is also a nitrogen-fixer.

Furthermore, B. amy produces a unique mixture of metabolites including antifungal, antiviral and antibacterial lipopeptide biosurfactants, organic acids such as propionic acid and acetic acid, and enzymes, such as amylases, lipases and proteases. For example, in some embodiments B. amy can produce a mixture of surfactin, fengycin, iturin, and lichenysin biosurfactants. In some embodiments B. amy can produce lipopeptides at amounts greater than other reference strains of B. amyloliquefaciens.

In certain embodiments, the composition can comprise B. subtilis B4. Strain B4 can produce lipopeptide biosurfactants in enhanced amounts, particularly surfactin. Advantageously, in some embodiments, B4 and/or the enhanced amounts of surfactin that it produces, can be especially helpful for enhanced disruption of methanogenic biofilms in livestock digestive tracts.

In some embodiments, B4 is “surfactant over-producing.” For example, the strain may produce at least 0.1-10 g/L, e.g., 0.5-1 g/L biosurfactant, or, e.g., at least 10%, 25%, 50%, 100%, 2-fold, 5-fold, 7.5 fold, 10-fold, 12-fold, 15-fold or more compared to other B. subtilis bacteria. For example, in some embodiments, ATCC 39307 can be used as a reference strain.

Thus, a combination of B. coagulans with B. amy and/or B4 can promote an enhanced rate of fermentation and direct control of microbial pests in a silage or grain storage environment.

In one embodiment, the composition comprises about 1×10³ to 1×10¹², 1×10⁴ to 1×10¹¹, 1×10⁵ to 1×10¹⁰, or 1×10⁶ to 1×10⁹ CFU/ml of B. coagulans.

In one embodiment, the composition comprises about 1×10³ to 1×10¹², 1×10⁴ to 1×10¹¹, 1×10⁵ to 1×10¹⁰, or 1×10⁶ to 1×10⁹ CFU/ml of B. amy.

In one embodiment, the composition comprises about 1×10³ to 1×10¹², 1×10⁴ to 1×10¹¹, 1×10⁵ to 1×10¹⁰, or 1×10⁶ to 1×10⁹ CFU/ml of B4.

In one embodiment, the composition comprises about 1×10³ to 1×10¹², 1×10⁴ to 1×10¹¹, 1×10⁵ to 1×10¹⁰, or 1×10⁶ to 1×10⁹ CFU/ml of another microorganism, such as a LAB.

In one embodiment, the composition can comprise a microbial inoculant comprising from 1 to 99% B. coagulans by weight or volume and from 99 to 1% B. amy by weight or volume of the inoculant. In some embodiments, the cell count ratio of B. coagulans to B. amy is about 1:9 to about 9:1, about 1:8 to about 8:1, about 1:7 to about 7:1, about 1:6 to about 6:1, about 1:5 to about 5:1 or about 1:4 to about 4:1.

In one embodiment, the composition can comprise a microbial inoculant comprising from 1 to 99% B. coagulans by weight or volume and from 99 to 1% B4 by weight or volume of the inoculant. In some embodiments, the cell count ratio of B. coagulans to B4 is about 1:9 to about 9:1, about 1:8 to about 8:1, about 1:7 to about 7:1, about 1:6 to about 6:1, about 1:5 to about 5:1 or about 1:4 to about 4:1.

In one embodiment, the composition can comprise a microbial inoculant comprising from 1 to 99% B. coagulans by weight or volume and from 99 to 1% total of B4 and B. amy by weight or volume of the inoculant. In some embodiments, the cell count ratio of B. coagulans to B4 and B. amy (combined total) is about 1:9 to about 9:1, about 1:8 to about 8:1, about 1:7 to about 7:1, about 1:6 to about 6:1, about 1:5 to about 5:1 or about 1:4 to about 4:1.

The species and ratio of microorganisms and other ingredients in the composition can be determined according to, for example, the type of silage or grain being treated, the humidity/moisture levels, and other environmental factors. Thus, the composition can be customizable for any given forage crop.

In certain embodiments, the silage additive composition comprises additional components, for example, carriers, dessicants, salts, dyes, flavors, buffers, nutrients, starches, carbohydrates, water, and/or additional microbial cultures and/or fermentation by-products.

In certain embodiments, the silage additive composition comprises diatomaceous earth, which can serve as, e.g., a pest control agent for silage pests including soft-bodied grubs, as well as pests that have exoskeletons. Diatomaceous earth comprises fossilized siliceous remains of a type of hard-shelled protest known as diatoms (e.g., Melosira and Monolithica). Microscopically, the particles are very sharp and can stick to an insect, becoming lodged between its exoskeletal joints. As the insect moves, its body receives cuts and lacerations from the sharp particles. Additionally, the abrasive particles can scratch away at the insects waxy exoskeletal layer, which then allows internal moisture to escape from the insect's body. Furthermore, soft-bodied pests can become dehydrated by diatomaceous earth, in addition to being cut by its abrasive edges.

The pests can be, for example, cockroaches, mites, beetles, borers, weevils, termites, ants, grasshoppers, aphids, slugs, snails, grubs, nematodes, and/or other worm-type or arthropod pests that may feed on the silage or stored grains.

Advantageously, diatomaceous earth can also serve as a hydroscopic substance, or desiccant, for reducing condensate formation in a silo or grain storage container, thereby making the environment less favorable for mold and fungal growth.

In preferred embodiments, the diatomaceous earth is in the form of a powder having a particle size of 1 micrometer to 1 millimeter, preferably from 10 to 200 micrometers. In some embodiments, pumice powder can also be used.

In certain embodiments, the composition comprises 0.01% to 75%, 0.1% to 50%, 0.5% to 25%, or 1.0% to 15% diatomaceous earth by weight.

In some embodiments, the silage additive composition comprises the fermentation products of one or more additional microorganisms, including, for example, beneficial yeasts, fungi and/or other bacteria. The fermentation product can contain a live and/or an inactive culture, purified or crude form growth by-products, such as biosurfactants, enzymes, and/or other metabolites, and/or any residual nutrients. The amount of biomass in the composition, by weight, may be, for example, anywhere from about 0.01% to 100%, about 1% to 90%, about 5% to about 80%, or about 10% to about 75%.

The fermentation product may be used directly, with or without extraction or purification. If desired, extraction and purification can be easily achieved using standard extraction and/or purification methods or techniques described in the literature.

In certain embodiments, the fermentation product is a product of one or more other Bacillus spp., such as, e.g., B. subtilis, B. licheniformis, B. mucilaginous, B. mojavensis, B. megaterium, and B. mucilaginosus.

In certain embodiments the fermentation product is a product of one or more yeasts and/or fungi, for example, Aureobasidium (e.g., A. pullulans), Blakeslea, Candida (e.g., C. apicola, C. bombicola, C. nodaensis), Cryptococcus, Debaryomyces (e.g., D. hansenii), Entomophthora, Hanseniaspora, (e.g., H. uvarum), Hansenula, Issatchenkia, Kluyveromyces (e.g., K. phaffii), Phycomyces, Pichia (e.g., P. anomala, P. guilliermondii, P. occidentalis, P. kudriavzevii), Pleurotus spp. (e.g., P. ostreatus), Pseudozyma (e.g., P. aphidis), Saccharomyces (e.g., S. boulardii sequela, S. cerevisiae, S. torula), Starmerella (e.g., S. bombicola), Torulopsis, Trichoderma (e.g., T. reesei, T. harzianum, T. hamatum, T. viride), Ustilago (e.g., U. maydis), Wickerhamomyces (e.g., W. anomalus, strain NRRL Y-68030), Williopsis (e.g., W. mrakii), Zygosaccharomyces (e.g., Z. bailii), and others.

In certain embodiments, the microorganisms utilized in the silage additive compositions have a number of beneficial properties that are useful for enhancing the ensiling process.

In one embodiment, the microorganisms are capable of producing a biosurfactant. As amphiphilic molecules, microbial biosurfactants reduce the surface and interfacial tensions between the molecules of liquids, solids, and gases. Furthermore, biosurfactants are biodegradable, have low toxicity, and can be produced using low cost and renewable resources. They can inhibit adhesion of undesirable microorganisms to a variety of surfaces, prevent the formation of biofilms, and can have powerful emulsifying and demulsifying properties.

Biosurfactants according to the subject methods can be selected from, for example, low molecular weight glycolipids (e.g., sophorolipids, cellobiose lipids, rhamnolipids, mannosylerythritol lipids and trehalose lipids), lipopeptides (e.g., surfactin, iturin, fengycin, arthrofactin and lichenysin), flavolipids, phospholipids (e.g., cardiolipins), and high molecular weight polymers such as lipoproteins, lipopolysaccharide-protein complexes, and polysaccharide-protein-fatty acid complexes.

The composition can comprise one or more biosurfactants at a concentration of 0.001% to 10%, 0.01% to 5%, 0.05% to 2%, and/or from 0.1% to 1% by weight.

In one embodiment, the silage additive composition further comprises one or more enzymes. The enzymes can be added in pure or crude form, and/or the enzymes can be produced by one or more microorganisms present in the silage additive composition. Enzymes can enhance breakdown of sugars and other polymers in forage, providing more easily fermentable compounds for LAB. Enzymes can also enhance the digestability of plant material for livestock animals.

Exemplary enzymes include, but are not limited to, phytases, amylases, proteases, lipases, xylanases, ferulic acid esterase, cellulase, hemicuellulases, pectinases, lignocellulytic enzymes, proteinase K, and others. In specific embodiments, the composition comprises a cellulase, protease, xylanase and/or amylase.

In some embodiments, the silage additive compositions comprise a carrier. Carriers can include liquid or solid carriers. For example, solid carriers may be made up of calcium carbonate, starch, baker's sugar, maltodextrin, cellulose and combinations thereof. Liquid carriers include, but are not limited to, water, fermentation broth from cultivation of a microbe, and/or a liquid nutrient medium.

Other additives to the subject compositions can include, for example, sodium silcoaluminate, sodium bicarbonate, dyes, dextrose, rehydration buffers, flavorings and/or silicon dioxide.

In certain embodiments, to improve or stabilize the effects of the composition, it can be blended with suitable adjuvants and then used as such or after dilution, if necessary. In preferred embodiments, the composition is formulated as a liquid, a concentrated liquid, or as dry powder or granules that can be mixed with water and other components to form a liquid product. In certain embodiments, the composition is formulated as, for example, liquid, dust, granules, microgranules, pellets, wettable powder, flowable powder, emulsions, microcapsules, oils, or aerosols.

Advantageously, the silage additive compositions according to the subject invention are non-toxic and can be applied in high concentrations without causing irritation to, for example, the skin or digestive tract of a human or other non-pest animal. Thus, the subject invention is particularly useful where application of the compositions occurs in the presence of living organisms, such as growers and livestock.

### Growth of Microbes for Producing Microbial Inoculants

The subject invention utilizes methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

As used herein “fermentation” of refers to cultivation or growth of cells under controlled conditions. The growth could be aerobic or anaerobic.

In one embodiment, the subject invention provides materials and methods for the production of biomass (e.g., viable cellular material), extracellular metabolites (e.g. small molecules and excreted proteins), residual nutrients and/or intracellular components (e.g. enzymes and other proteins).

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In some embodiments, the method for cultivation may further comprise adding additional acids and/or antimicrobials in the medium before, and/or during the cultivation process. Antimicrobial agents or antibiotics are used for protecting the culture against contamination.

Additionally, antifoaming agents may also be added to prevent the formation and/or accumulation of foam during submerged cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%/0, 80%, or 90%.

The microbial growth by-product produced by microorganisms of interest may be retained in the microorganisms or secreted into the growth medium. The medium may contain compounds that stabilize the activity of microbial growth by-product.

The biomass content of the fermentation medium may be, for example, from 5 g/l to 180 g/l or more, or from 10 g/l to 150 g/1.

The cell concentration may be, for example, at least 1×10⁶ to 1×10¹², 1×10⁷ to 1×10¹¹, 1×10⁸ to 1×10¹⁰, or 1×10⁹ CFU/ml.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

The microbial inoculants produced may be used without further stabilization, preservation, and storage, however. Advantageously, direct usage of these microbial inoculants preserves a high viability of the microorganisms, reduces the possibility of contamination from foreign agents and undesirable microorganisms, and maintains the activity of the by-products of microbial growth.

In other embodiments, the composition (microbes, growth medium, or microbes and medium) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 pint to 1,000 gallons or more. In certain embodiments the containers are 1 gallon, 2 gallons, 5 gallons, 25 gallons, or larger.

### Preparation of Microbe-Based Products

The subject invention provides “microbe-based products,” which are products that are to be applied in practice to achieve a desired result. The microbe-based product can be simply a microbial inoculant harvested from the microbe cultivation process. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers and/or agents that facilitate tracking of the microbes and/or the composition in the environment to which it is applied. The microbe-based product may also comprise mixtures of microbial inoculants. The microbe-based product may also comprise one or more components of a microbial inoculant that have been processed in some way such as, but not limited to, filtering, centrifugation, lysing, drying, purification and the like.

One microbe-based product of the subject invention is simply the fermentation medium containing the microorganisms and/or the microbial metabolites produced by the microorganisms and/or any residual nutrients. The product of fermentation may be used directly without extraction or purification. If desired, extraction and purification can be easily achieved using standard extraction and/or purification methods or techniques described in the literature.

The microorganisms in the microbe-based products may be in an active or inactive form, or in the form of vegetative cells, reproductive spores, conidia, mycelia, hyphae, or any other form of microbial propagule. The microbe-based products may also contain a combination of any of these forms of a microorganism.

In one embodiment, different strains of microbe are grown separately and then mixed together to produce the microbe-based product. The microbes can, optionally, be blended with the medium in which they are grown and dried prior to mixing.

In one embodiment, the different strains are not mixed together, but are applied to a plant and/or its environment as separate microbe-based products.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

Upon harvesting the microbe-based composition from the growth vessels, further components can be added as the harvested product is placed into containers or otherwise transported for use. The additives can be, for example, buffers, carriers, other microbe-based compositions produced at the same or different facility, viscosity modifiers, preservatives, nutrients for microbe growth, surfactants, emulsifying agents, lubricants, solubility controlling agents, tracking agents, solvents, biocides, antibiotics, pH adjusting agents, chelators, stabilizers, ultra-violet light resistant agents, other microbes and other suitable additives that are customarily used for such preparations.

In one embodiment, buffering agents including organic and amino acids or their salts, can be added. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

The pH of the microbe-based composition should be suitable for the microorganism(s) of interest. In a preferred embodiment, the pH of the composition is about 3.5 to 7.0, about 4.0 to 6.5, or about 5.0.

In one embodiment, glucose, glycerol and/or glycerin can be added to the microbe-based product to serve as, for example, an osmoticum during storage and transport. In one embodiment, molasses can be included.

In one embodiment, prebiotics can be added to and/or applied concurrently with the microbe-based product to enhance microbial growth. Suitable prebiotics, include, for example, kelp extract, fulvic acid, chitin, humate and/or humic acid.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., a silo). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This allows for a scaled-down bioreactor (e.g., smaller fermentation vessel, smaller supplies of starter material, nutrients and pH control agents), which makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

Advantageously, the compositions can be tailored for use at a specified location. In one embodiment, the microbe growth facility is located on, or near, a site where the microbe-based products will be used (e.g., a citrus grove).

Advantageously, these microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products to improve agricultural production.

The cultivation time for the individual vessels may be, for example, from 1 to 7 days or longer. The cultivation product can be harvested in any of a number of different ways.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### Methods of Producing Silage

In preferred embodiments, methods for ensiling forage are provided, wherein the method comprises inoculating forage with a silage additive composition according to the subject invention, and wherein the microbial inoculant(s) of the composition grow anaerobically, thereby fermenting the forage and producing silage.

Inoculation can comprise applying the composition to the forage and, optionally, mixing the composition with the forage. Preferably, the forage is contained in an airtight silage bag, tarp, silo or tank, which can be sealed after inoculation with optional mixing so that anaerobic fermentation can occur.

To improve or stabilize the effects of the treatment composition, it can be blended with suitable adjuvants and then used as such or after dilution if necessary. In preferred embodiments, the composition is formulated as a dry powder or as granules, which can be mixed with water and other components to form a liquid product.

In certain embodiments, the silage additive composition is applied directly to the forage in dried form, after which a source of liquid and/or nutrient medium is applied to the mixture to create an environment conducive to fermentation. In certain other embodiments, when the forage has a sufficient moisture content (e.g., 45% to 90% moisture content by weight), the silage additive composition can be applied in dried form without the addition of a liquid. In yet other embodiments, the silage additive is pre-mixed with water or a nutrient medium before application to the forage.

In certain embodiments, the method comprises applying at least 20 billion, at least 30 billion, at least 40 billion CFU, at least 50 billion CFU, at least 60 billion CFU, at least 70 billion CFU, at least 80 billion CFU, at least 90 billion CFU, or at least 100 billion CFU of the microbial inoculant(s) per ton of forage. In certain specific embodiments, the method comprises applying about 100,000 CFU/g of forage.

In some embodiments, fermentation proceeds for a period of time ranging from, for example, 24 hours to 12 weeks, 36 hours to 10 weeks, 48 hours to 8 weeks, 56 hours to 6 weeks, or 72 hours to 4 weeks. In an exemplary embodiment, the fermentation period is about 10 days to 3 weeks. The methods can comprise sampling the silage to, for example, monitor the pH throughout the fermentation period and/or to check for potential contamination of the silage.

In some embodiments, the methods further comprise applying materials with the composition to enhance microbe growth during application (e.g., nutrients, germination enhancers and/or prebiotics to promote microbial germination and/or growth). In one embodiment, nutrient sources can include, for example, sources of nitrogen, potassium, phosphorus, magnesium, proteins, vitamins and/or carbon. In one embodiment, prebiotics can include, for example, kelp extract, fulvic acid, chitin, humate and/or humic acid. In one embodiment, germination enhancers include L-alanine and/or L-valine.

In one embodiment, application of the silage additive compositions according to the method controls enhances the rate of silage fermentation.

In one embodiment, application of the silage additive compositions according to the method results in direct control of molds, fungi and bacteria that can cause spoilage of silage and/or stored grains.

In one embodiment, application of the silage additive compositions according to the method results in direct control of arthropods, worms, slugs, snails, grubs and other pests that feed on and/or infect silage and/or stored grains.

In one embodiment, application of the silage additive compositions according to the method enhances the nutritional benefits of the silage and/or stored grains as a feed source for livestock.

In some embodiments, the subject invention provides methods of controlling stored grain pests, such as, for example, grain borers. weevils, moths, and beetles, wherein a silage additive composition of the subject invention is applied to the stored grain. The grain can include, for example, flour, maida, suji, beans, cotton seeds, corn, rice, barley, amaranth, buckwheat, bulgur wheat, farro, flax, kamut, millet, quinoa, rye, spelt, teff, and triticale.

In some embodiments, the subject invention provides methods of feeding a livestock animal, wherein silage that has been produced according to embodiments of the subject invention is made available to the livestock animal so that the animal may ingest the silage. Advantageously, the feed source can provide health benefits to the livestock animal, including, for example, improved growth rate (e.g., average daily gain), improved feed-to-muscle conversion, improved milk quality and quantity, increased lifespan, improved immune health, improved gut flora, reduced digestive pathogens, and reduced enteric methane production.

“Livestock” animals, as used herein, are “domesticated” animals, meaning species that have been influenced, bred, tamed, and/or controlled over a sustained number of generations by humans, such that a mutualistic relationship exists between the animal and the human. Particularly, livestock animals include animals raised in an agricultural or industrial setting to produce commodities such as food, fiber and labor. Types of animals included in the term livestock can include, but are not limited to, alpacas, llamas, pigs (swine), horses, mules, asses, camels, dogs, ruminants, chickens, turkeys, ducks, geese, guinea fowl, and squabs.

In certain embodiments, the livestock animals are “ruminants,” or mammals that utilize a compartmentalized stomach suited for fermenting plant-based foods prior to digestion with the help of a specialized gut microbiome. Ruminants include, for example, bovines, sheep, goats, ibex, giraffes, deer, elk, moose, caribou, reindeer, antelope, gazelle, impala, wildebeest, and some kangaroos.

In specific exemplary embodiments, the livestock animals are bovine animals, which are ruminant animals belonging to the subfamily Bovinae, of the family Bovidae. Bovine animals can include domesticated and/or wild species. Specific examples include, but are not limited to, water buffalo, anoa, tamaraw, auroch, banteng, guar, gayal, yak, kouprey, domestic meat and dairy cattle (e.g., Bos taurus, Bos indicus), ox, bullock, zebu, saola, bison, buffalo, wisent, bongo, kudu, kewwel, imbabala, kudu, nyala, sitatunga, and eland.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Solid State Fermentation of Bacillus Microbes

For Bacillus spp. spore production, a wheat bran-based media is used. The media is spread onto stainless steel pans in a layer about 1 to 2 inches think and sterilized.

Following sterilization, the pans are inoculated with seed culture. Optionally, added nutrients can be included to enhance microbial growth, including, for example, salts and/or carbon sources such as molasses, starches, glucose and sucrose. To increase the speed of growth and increase the motility and distribution of the bacteria throughout the culture medium, potato extract or banana peel extract can be added to the culture.

Spores of the Bacillus strain of choice are then sprayed or pipetted onto the surface of the substrate and the trays are incubated between 32-40° C. Ambient air is pumped through the oven to stabilize the temperature. Incubation for 48-72 hours can produce 1×10¹⁰ spores/gram or more of the strain.

### Example 2—Co-Cultivation of B. amy and M. xanthus for Enhanced Lipopeptide Production

In one embodiment, compositions comprising lipopeptides (e.g., surfactin, iturin and/or fengycin) are produced using co-cultivation of B. amy and Myxococcus xanthus. When grown together, the species try to inhibit one another, thereby producing high concentrations of lipopeptides.

B. amy inoculum is grown in a small-scale reactor for 24 to 48 hours. Myxococcus xanthus inoculum is grown in a 2 L working volume seed culture flask for 48 to 120 hours. A fermentation reactor is inoculated with the two inocula. The nutrient medium comprises:

| | |
|---|---|
| Glucose | 1 g/L to 5 g/L |
| Casein peptone | 1 g/L to 10 g/L |
| K₂HPO₄ | 0.01 g/L to 1.0 g/L |
| KH₂PO₄ | 0.01 g/L to 1.0 g/L |
| MgSO₄•7H₂O | 0.01 g/L to 1.0 g/L |
| NaCl | 0.01 g/L to 1.0 g/L |
| CaCO₃ | 0.5 g/L to 5 g/L |
| Ca(NO₃)₂ | 0.01 g/L to 1.0 g/L |
| Yeast extract | 0.01 g/L to 5 g/L |
| MnCl₂•4H₂O | 0.001 g/L to 0.5 g/L |
| Teknova trace element | 0.5 ml/L to 5 ml/L |

Fine grain particulate anchoring carrier is suspended in the nutrient medium. The carrier comprises cellulose (1.0 to 5.0 g/L) and/or corn flour (1.0 to 8.0 g/L).

The B. amy produces lipopeptides into the liquid fermentation medium. The entire culture can be used as-is, or the culture can be processed and, optionally, the lipopeptides purified.

### Example 3—B. amy as Livestock Feed Enhancer

B. amy is particularly advantageous over traditional probiotic microorganisms due to its ability to produce spores that remain viable in the digestive tract and, in some embodiments, after excretion in the animal's waste. Additionally, B. amy produces a unique mixture of metabolites that provide a broad-spectrum of digestive and environmental benefits when administered to a livestock animal.

In certain embodiments, as exemplified in Table 1 below, the growth by-products can directly inhibit methanogens, disrupt methanogen biofilms, and/or reduce H₂ concentration in a livestock animal's digestive system.

TABLE 1
Exemplary B. amy growth by-products for reducing methanogenesis and H₂

| | Growth | |
|---|---|---|
| Function(s) | by-product(s) | Examples (Produced by B. amy) |
| Inhibition of | Enzymes | Proteinase K (and/or a homolog thereof): can |
| methanogens | | specifically lyse pseudomurien, a major structural cell |
| | | wall component of some archaea, including methanogens. |
| | | Diglycolic acid dehydrogenase (DGADH), (and/or a |
| | | homolog thereof): can disrupt ether bonds between the |
| | | glycerol backbone and fatty acids of the phospholipid |
| | | layer of archaeal cell membranes. |
| | Organic acids | Propionic acid and/or acetic acid: can disrupt the |
| | | structure of archaeal cell membranes. |
| Disruption of | Lipopeptide | Surfactin, fengycin, iturin, bacillomycin, lichenysin, |
| methanogen | biosurfactants; | difficidin, and/or a maltose-based glycolipid: can |
| biofilms | Glycolipid | interfere with the production and/or maintenance of the |
| | biosurfactants; | exopolysaccharide matrix that forms biofilms, thereby |
| | Polyketides | interfering with formation and/or adhesion of |
| | | capabilities of the biofilm. |
| Reduction of | Organic acids | Propionic acid: can stimulate acetogenic |
| H₂ | | microorganisms, which produce acetic acid from |
| | | hydrogen and carbon dioxide. This results in reduced |
| | | hydrogen availability for methanogenic microbes to |
| | | carry out methanogenesis, and also helps keep H₂ |
| | | concentrations from increasing when methanogenesis |
| | | decreases. Increased H₂ can lead to a build-up of |
| | | trimethylamine in the digestive system, which causes a |
| | | “fishy” smell in produced milk. |

In one embodiment, as exemplified in Table 2 below, the composition comprises B. amy, and/or growth by-products thereof, which can enhance the overall health and productivity of a livestock animal by performing a variety of health-promoting functions. Thus, in some embodiments, B. amy can serve as a probiotic when administered to an animal.

TABLE 2
Exemplary B. amy growth by-products for enhancing livestock health

| | Growth | |
|---|---|---|
| Function(s) | by-product(s) | Examples (Produced by B. amy) |
| Regulation of | Biosurfactants; | Biosurfactants, including lipopeptides and |
| gut microbiome | Natural antibiotics; | glycolipids, as well as natural antibiotics (e.g., |
| | Organic acids | polyketides, penicillins, cephalosporins, |
| | | validamycins, carbapanems, and nocardicins): can |
| | | inhibit the growth of pathogenic, or otherwise |
| | | deleterious gut microorganisms (e.g., Anaeroplasma, |
| | | Acholeplasma and certain fungi) by, for example, |
| | | interfering with the pathogenic or deleterious |
| | | microorganism's cell membrane and/or biofilm structure. |
| | | Organic acids, such as propionic acid: can promote |
| | | the growth of beneficial gut microorganisms (e.g., |
| | | Proteobacteria, Rhodospirillaceae, Campylobacterales |
| | | and Butyricimonas) by, for example, altering the pH |
| | | of the digestive system to a more favorable |
| | | environment for such growth. |
| | | In certain embodiments, regulation of the gut |
| | | microbiome also leads to a reduction in nitrous |
| | | oxide emissions due to a reduction in |
| | | ammonia-oxidizing gut bacteria. |
| Stimulation of | Organic acids; | Organic acids, such as the short-chain fatty acids |
| growth hormones | Biosurfactants; | butyrate and valerate: can improve digestion through, |
| (e.g., GH/IGH-1); | Digestive enzymes | for example, improved intestinal and/or ruminal cell |
| increasing the rate | | function. |
| of weight gain; | | Lipopeptide and glycolipid biosurfactants: can |
| and increasing | | improve digestion by, for example, enhancing the |
| feed-to-muscle | | bioavailability of nutrients and water through |
| conversion through | | intestinal/ruminal cells and improve absorption |
| improved digestion | | thereof into the bloodstream. |
| | | Digestive enzymes, such as amylases, lipases, and |
| | | proteases (e.g., collagenase-like protease, peptidase E |
| | | (N-terminal Asp-specific dipeptidase), peptidase s8 |
| | | (subtilisin-like serine peptidase), serine peptidase, |
| | | and endopeptidase La): can improve conversion of |
| | | feed to muscle by increasing digestion of proteins, |
| | | fats and carbohydrates in feed that can otherwise be |
| | | difficult or impossible for the animal to digest. |
| | | Additionally, because nitrogen is required for |
| | | conversion of feed to muscle mass, increased |
| | | nitrogen uptake in the digestive system due to |
| | | improved muscle conversion can result in fewer |
| | | nitrous oxide precursors, and accordingly, fewer |
| | | nitrous oxide emissions. |
| Improving quantity | Lignocellulytic | Lignocellulytic enzymes, such as cellulose, xylanase, |
| and quality of | enzymes; | laccase, and manganese catalase: can enhance |
| produced milk in | Folic acid/folate | digestion of polysaccharides, such as cellulose, |
| mammals | | xylan, hemicellulose, and lignin, into the components |
| | | necessary for milk production. |
| | | Folate: can help increase milk production by, for |
| | | example, enhancing mammary gland metabolism. |
| | | Additionally, folate is an important nutrient for, e.g., |
| | | growth and neural development. Thus, increased |
| | | folate in produced milk can improve the nutritional |
| | | quality of the milk for nursing offspring, thereby |
| | | potentially shortening the time required for weaning |
| | | and/or increasing the growth and survival rate of |
| | | offspring. |
| Enhancing immune | Vitamins | Riboflavin, produced via riboflavin synthase: can |
| health, life | | provide antinociception and anti-inflammatory |
| expectancy and | | effects in a livestock animal. |
| overall health | | Folate, produced via bifunctional folate synthesis |
| | | protein: can help regulate energy conversion, gene |
| | | expression and DNA production, in addition to being |
| | | an anti-inflammatory agent. |
| | | Ubiquinone (CoQ10), produced via ubiquinone |
| | | biosynthesis O-methyltransferase: can, as an |
| | | antioxidant, prevent low-density lipoprotein |
| | | oxidation, which can result in atherosclerosis. |

1. A silage additive composition comprising a microbial inoculant, diatomaceous earth, and, optionally, a carrier,
wherein, the microbial inoculant comprises one or more strains of bacteria and/or a growth by-product thereof, and
wherein at least one of the one or more strains of bacteria is capable of producing lactic acid in the presence of forage.

2. The silage additive composition of claim 1, wherein the strain of bacteria capable of producing lactic acid is Bacillus coagulans.

3. The silage additive composition of claim 1, wherein the microbial inoculant comprises B. amyloliquefaciens strain NRRL B-67928 (“B. amy”).

4. The silage additive composition of claim 1, wherein the microbial inoculant comprises B. subtilis strain NRRL B-68031 (“B4”).

5-6. (canceled)

7. The silage additive composition of claim 1, further comprising a fermentation product of one or more additional beneficial microorganisms selected from Wickerhamomyces anomalus, Starmerella bombicola, Saccharomyces chlororaphis, Saccharomyces cerevisiae, Pleurotus ostreatus, Debaryomyces hansenii, and Meyerozyma guilliermondii.

8. (canceled)

9. A method for ensiling forage, wherein the method comprises inoculating forage contained in a silage environment with a silage additive composition comprising a microbial inoculant and, optionally, a carrier,
wherein, the microbial inoculant comprises one or more strains of bacteria and/or a growth by-product thereof,
wherein at least one of the one or more strains of bacteria is capable of producing lactic acid, and
wherein the microbial inoculant grows anaerobically in the silage environment and ferments the forage to produce silage.

10. The method of claim 9, wherein the forage comprises cuttings of grass, maize, sorghum, cereal, oat and/or alfalfa plants.

11. (canceled)

12. The method of claim 9, wherein the composition is applied directly to the forage and, optionally, mixed with the forage, and wherein the silage environment is sealed to produce an anaerobic environment.

13. The method of claim 9, wherein the microbial inoculant ferments the forage for a period of time ranging from about 24 hours to 12 weeks.

14. The method of claim 9, wherein the strain of bacteria capable of producing lactic acid is Bacillus coagulans.

15. The method of claim 9, wherein the microbial inoculant comprises Bacillus amyloliquefaciens “B. amy” and a growth by-product thereof.

16. The method of claim 9, wherein the microbial inoculant comprises Bacillus subtilis “B4” and a growth by-product thereof.

17. The method of claim 9, wherein the growth by-product is an organic acid, a biosurfactant and/or an enzyme.

18. (canceled)

19. The method of claim 9, further comprising a fermentation product of one or more additional beneficial microorganisms selected from Wickerhamomyces anomalus, Starmerella bombicola, Saccharomyces chlororaphis, Saccharomyces cerevisiae, Pleurotus ostreatus, Debaryomyces hansenii, and Meyerozyma guilliermondii.

20. The method of claim 19, wherein the fermentation product comprises purified or crude form growth by-products of the beneficial microorganism, fermentation medium, cells, and/or nutrients residual from fermentation.

21. The method of claim 9, wherein the composition further comprises diatomaceous earth.

22. A method for controlling stored grain pests, wherein a silage additive composition according to claim 1 is applied to the stored grain.

23. The method of claim 22, wherein the stored grain is flour, maida, suji, beans, cotton seeds, corn, rice, barley, amaranth, buckwheat, bulgur wheat, farm, flax, kamut, millet, quinoa, rye, spelt, teff, and/or triticale.

24. The method of claim 22, wherein the stored grain pest is a grain borer, weevil, moth, and/or beetle.

25. A method of feeding a livestock animal, the method comprising producing silage using a method according to claim 9, and making the silage available to the livestock animal so that the animal may ingest the silage.
